(19) United States
(12) Patent Application Publication
(10) Pub. No.: US 2005/0058714 A1
(43) Pub. Date: Mar. 17, 2005

(54) Dispenser for the selective release of microparticles

(76) Inventors: David J. Beebe, Monona, WI (US); Glennys A. Mensing, Middleton, WI (US); David T. Eddington, Madison, WI (US)

Correspondence Address: Peter C. Stomma;BOYLE, FREDRICKSON, NEWHOLM, STEIN & GRATZ S.C., Suite 1030, 250 E. Wisconsin Avenue, Milwaukee, WI, 53202, US

(21) Appl. No.: 10/663,126

(22) Filed: Sep. 16, 2003

(51) Int. Cl. A61M 31/00; A61K 9/14

(52) U.S. Cl. 424/489; 604/57

(57) ABSTRACT

A microparticle storage and delivery device is provided for allowing an individual to selectively dispense a dosage of a microparticle. The device includes a hydrogel string loaded with a plurality of dosages of the microparticle and a storage container for receiving the string. The storage container has a first open end and a second opposite end. A biasing structure urges the string toward the open end of the storage container and an actuator selectively engages the string to urge a dosage of the microparticle from the open end of the storage container.

### REFERENCE TO GOVERNMENT GRANT

This invention was made with United States government support awarded by the following agencies: DOD/ARPA F30602-00-2-0570. The United States has certain rights in this invention.

### FIELD OF THE INVENTION

This invention relates generally to medical devices, and in particular, to a dispenser implantable in a human body that allows for a user to selectively dispense microsize drug particles into the body.

### BACKGROUND AND SUMMARY OF THE INVENTION

As is known, many individuals take various types of drugs and/or medications to treat corresponding physical conditions. Various factors must be considered when administering such medication. Specifically, the medication must be supplied to the body in the correct amount and in a timely manner. Further, it is often required that the medication be directed to a specific area of the body to maximize the beneficial effects. By way of example, the timely delivery of medication such as Sildenafil Citrate to the lumen of the penis is required for proper treatment of erectile dysfunction.

During sexual arousal, the arteries in the penis expand so as to allow more blood to flow therein. Simultaneously, as the arteries expand, the veins that normally carry blood away from the penis become compressed thereby restricting the blood flow therefrom. It can be appreciated that as more blood flows into the penis then flows out, the penis will enlarge thereby resulting in an erection. In situations where erectile dysfunction occurs, insufficient blood flows to the penis. In order to combat erectile dysfunction, Sildenafil Citrate is often prescribed.

Sildenafil Citrate causes the arteries in the penis to expand thereby increasing the blood flow thereto. Presently, Sildenafil Citrate is taken in a pill form by the individual experiencing erectile dysfunction. It can be appreciated that the pill must go through the digestive track of the individual in order to be absorbed into the individual's blood stream. Often times, it will take over an hour or more for the Sildenafil Citrate pill to take effect.

Alternatively, soft tabs have been developed that may be absorbed into the blood stream of an individual through the cell walls under the individual's tongue. As a result, since the Sildenafil Citrate does not have to pass through the digestive track into the blood stream, the time period for the Sildenafil Citrate to take effect can be greatly reduced. However, it will still take approximately fifteen (15) minutes before the effects of the Sildenafil Citrate are realized. Therefore, it is highly desirable to provide a method or device that allows for the reliable delivery of microparticles of a medication directly to a desired area of a human body.

Therefore, it is a primary object and feature of the present invention to provide a dispenser implantable within a human body for selectively releasing medication on demand.

It is a further object and feature of the present invention to provide a dispenser implantable within a human body for selectively releasing medication on demand that is simple to utilize and inexpensive to manufacture.

It is a still further object and feature of the present invention to provide a dispenser implantable within a human body for selectively releasing medication on demand that may be simply and easily refilled within the body.

In accordance with the present invention, a drug storage and delivery device is provided for allowing an individual to selectively dispense a dosage of a microparticle. The device includes a string loaded with a plurality of dosages of the microparticle and a storage container for receiving the string. The storage container has a first open end and a second opposite end. A biasing structure is provided for urging the string toward the open end. An actuator selectively engages the string to urge a dosage of the microparticle from the open end of the storage container.

The string includes a hydrogel having the plurality of dosages of the microparticle contained therein. The device includes an hollow actuator housing having an interior for slidably receiving the actuator therein. The actuator includes a staging section and a porous section. The open end of the storage container communicates with the staging section of the interior of the actuator housing. A first valve isolates the porous section from the staging section of the interior of the actuator housing. A second valve selectively closes an open end of the actuator housing.

A non-porous plug may be disposed in the storage container between the string and the biasing structure. Further, the second end of the storage container is porous. The biasing structure includes a hydrogel element that expands in response to a fluid passing through the second end of the storage container.

In accordance with a further aspect of the present invention, a dispenser is provided that is implantable in the human body for allowing a user to selectively dispense a dosage of a microparticle. The dispenser includes a storage container for receiving a plurality of dosages of the microparticle therein. An actuator is operatively connected to the storage container. The actuator releases a single dosage of the microparticle from the storage container in response to actuation by a user.

The actuator may include a series of hydrogel triggers. One of the triggers expands in response to actuation by the user. The dispenser may also include a plunger operatively engageable with the plurality of dosages. The plunger urges a single dosage from the storage container in response to expansion of one of the hydrogel triggers. Alternatively, the dispenser may include a string formed from a hydrogel. The hydrogel has a plurality of dosages of the microparticle contained therein. The biasing structure urges the string from the storage container. It is contemplated to provide a non-porous plug in the storage container between the string and the biasing structure.

The dispenser may include a hollow actuator housing having an interior for slidably receiving the actuator therein. The storage container has a first end communicating with the interior of the actuator housing and a second end. The interior of the actuator housing includes a staging section and a porous section. The storage container communicates with the staging section of the interior of the actuator housing. A first valve isolates the porous section from the staging section of the interior of the actuator housing. A second valve selectively closes an open end of the actuator housing. It is contemplated that the second end of the storage container be porous.

In accordance with a still further aspect of the present invention, a method is provided for dispensing a dosage of a microparticle to an individual. The method includes the steps of providing a dispenser and loading the dispenser with a first plurality of dosages of the microparticle. The dispenser is implanted in a body and one of the first plurality of dosages is selectively released.

The first plurality of dosages of the microparticle may be encapsulated in a hydrogel. The dispenser includes a storage container for receiving the first plurality of dosages therein. It is contemplated that the step of selectively releasing one of the first of a plurality of dosages include the additional step of urging a single dosage from the storage container. It is further contemplated to isolate the first plurality of dosages of the microparticle in the dispenser from the interior of the body after the step of implanting the dispenser in the body. After the first plurality of dosages is released into the body, the dispenser may be loaded with a second plurality of dosages of the microparticle.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is a schematic view of a dispenser in accordance with the present invention in a non-actuated position;

FIG. 2 is a schematic view of the dispenser of FIG. 1 in an actuated position;

FIG. 3 is a schematic view of a container for housing a string to be received in the dispenser of FIG. 1;

FIG. 4 is a schematic view of an alternate embodiment of the dispenser of the present invention in a non-actuated position;

FIG. 5 is a schematic view, similar to FIG. 4, showing the dispenser in a first actuated position;

FIG. 6 is a schematic view, similar to FIG. 4, showing the dispenser in a second actuated position;

FIG. 7 is a schematic view of a third embodiment of a dispenser in accordance with the present invention in a non-actuated position;

FIG. 8 is a schematic view, similar to FIG. 7, showing the dispenser in a first actuated position; and

FIG. 9 is a schematic view, similar to FIG. 7, showing the dispenser in a second actuated position.

### DETAILED DESCRIPTION OF THE DRAWINGS

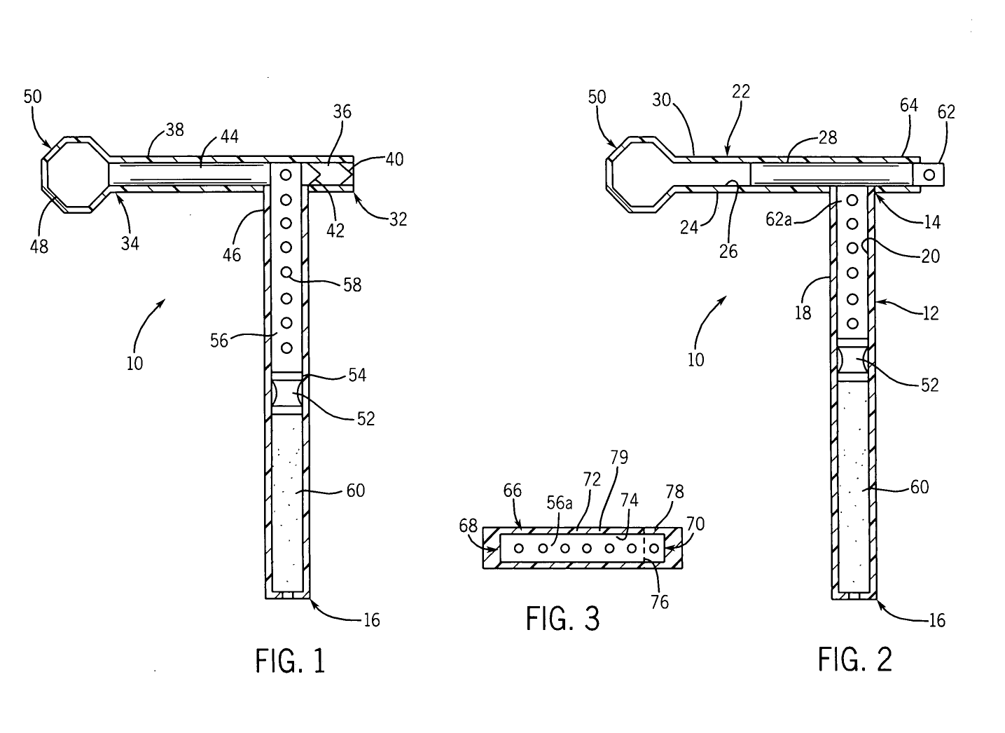

Referring to FIGS. 1-2, a dispenser in accordance with the present invention is generally designated by the reference numeral 10. It is intended that the dispenser 10 be implantable within a human body, for reasons hereinafter described. Dispenser 1O includes a generally hollow storage container 12 having a first open end 14 and a second porous end 16. Storage container 12 includes a generally tubular wall 18 having an inner surface 20 defining a storage cavity within storage container 12.

Open end 14 of storage container 12 communicates with the interior of actuator housing 22. Actuator housing 22 includes a tubular wall 24 having an inner surface 26 that defines the interior of actuator housing 22. Inner surface 26 forms a slidable interface with the outer surface 28 of actuator 30, for reasons hereinafter described. Actuator housing 22 further includes a first open end 32 and an opposite actuating end 34. The interior of actuator housing 22 includes a first porous portion 36 and a second staging portion 38. Porous portion 36 of the interior of actuator housing 22 is isolated from the exterior of dispenser 10 by unidirectional valve 40 that prevents fluids from outside of dispenser 10 from entering the interior of actuator housing 22. In addition, staging portion 38 of the interior of actuator housing 22 is isolated from porous portion 36 of the interior of actuator housing 22 by unidirectional valve 42 that prevents fluids from within porous portion 36 from entering the interior of staging portion 38.

Solid actuator 44 is slidably received within the interior of actuator housing 22. Actuator 44 includes a first terminal end 46 and an actuating end 48 that extends through end 34 of actuator housing 22 into a bulbous actuating element 50 mounted on second end 34 of actuator housing 22. As hereinafter described, actuator 44 is movable between a non-actuated position, FIG. 1, and an actuated position, FIG. 2, in response to compression of bulbous actuating element 50.

A non-porous plug 52 is positioned within the storage cavity within the interior of storage container 12. Plug 52 includes outer surface 54 that forms a slidable interface with inner surface 20 of storage container 12. Plug 52 separates the storage cavity within storage container 12 into a first portion for receiving a string 56 loaded with a plurality of dosages 58 of a microparticle and a second portion for receiving a biasing structure such as expandable hydrogel 60. By way of example, string 56 may take the form of a viscous fluid encapsulating the dosages; a hydrogel string embedded with the dosages; or multiple capsules having corresponding dosages disposed in each. In addition, string 58 may take other forms without deviating from the scope of the present invention. Hydrogel 60 extends between porous end 16 of storage container 12 and plug 52 to urge plug 52, and hence string 56, towards actuator housing 22.

In operation, dispenser 10 is implanted in the human body at a location wherein release of a dosage of the microparticle would be most beneficial to a patient. With actuator rod 44 in a non-actuated position, FIG. 1, hydrogel 60 urges a portion 62 of string 56 into staging portion 38 of the interior of actuator housing 22 through open end 14 of storage container 12. It is contemplated that hydrogel 60 expand in response to pressure from bodily fluids that are allowed to pass through porous end 16 of storage container 12.

In response to compression of bulbous actuating element 50 by a user, terminal end 46 of actuator rod 44 is urged toward its actuating position, FIG. 2. As actuator rod 44 moves from its non-actuated position, FIG. 1, to its actuator position, FIG. 2, actuator rod 44 shears off a predetermined portion 62 of string 56 and urges portion 62 of string 56 through unidirectional valves 42 and 40 into the human body. Thereafter, bulbous actuating element 50 is released by the user such that actuator rod 44 returns to its non-actuated position, FIG. 1. It is contemplated that a portion 64 of wall 24 of actuator housing 22 that defines porous portion 36 be porous so as to allow actuator rod 44 to return to its non-actuating position, FIG. 1, without undue pressure build-up. Once actuator rod 44 has returned to its non-actuated position, FIG. 1, hydrogel 60 expands under pressure from the bodily fluids allowed into storage container 12 through porous end 16 and urges second portion 62a of string 58 into the staging portion 38 of the interior of actuator housing 22. It can be appreciated that subsequent portions of the string may be dispensed into the body in the same manner as portion 62 of string 58, as heretofore described.

While string 56 has been described as a uniform hydrogel string loaded with microparticles, the string may take other forms without deviating from the scope of the present invention. For example, each portion of string 56 may be separated from the adjacent portion to facilitate movement of a portion of string 58 from the staging portion 38 of the interior of actuator housing 22 into the body. Further, the microparticles within string 56 are depicted as single elements. However, it is contemplated that the microparticles constitute a plurality of microscale particles disposed throughout portion 62 of hydrogel string 58. By way of example, microparticles 58 within hydrogel string 56 may take the form of drug capsules, cells, oocytes, embryos or microparts.

Once string 58 has been completely dispensed into the body, it is contemplated to replace both string 56 and hydrogel 60 in storage container 12 while dispenser 10 remains in the body. By way of example, a syringe may be used to inject a replacement string 56 into storage container 12. Similarly, a syringe may be used to either remove a portion of hydrogel 60 or to replace the same in order to maintain sufficient biasing force on string 56 to urge a portion thereof into staging portion 38 of the interior of actuator housing 22.

Referring to FIG. 3, a container housing replacement string 56a is generally designated by the reference numeral 66. Container 66 includes first and second closed ends 68 and 70, respectively, interconnected by tubular wall 72. Inner surface 74 of tubular wall 72 defines a cavity for receiving replacement string 56a therein. It is contemplated to provide perforations 76 in tubular wall 72 that allows first portion 78 of container 66 to be removed from main portion 79 of container 66 with minimal mechanical force in order to expose a portion of replacement string 56a. Thereafter, replacement string 56a may be used to reload storage container 12 of dispenser 10, as heretofore described.

Referring to FIGS. 4-6, an alternate embodiment of a dispenser in accordance with the present invention is generally designated by the reference numeral 80. Dispenser 80 includes an expandable actuator 82 having first and second opposite ends 84 and 86, respectively. Actuator 82 includes a plurality of expandable sections, 88a-g. Each expandable section 88a-g of actuator 82 includes a corresponding expandable hydrogel trigger 90a-g positioned therein. End 84 of actuator 82 is rigidly connected to end 92 of plunger 94. Second end 96 of plunger 94 is operatively connected to a lower end of inner sheath 98. Inner sheath 98 is telescopically received within outer sheath 100. In a non-actuated position, upper end 104 of outer sheath 100 is generally co-planar with upper end 102 of inner sheath 98. A plurality of dosages 106a-g of microparticles is disposed within inner sheath 98, for reasons hereinafter described.

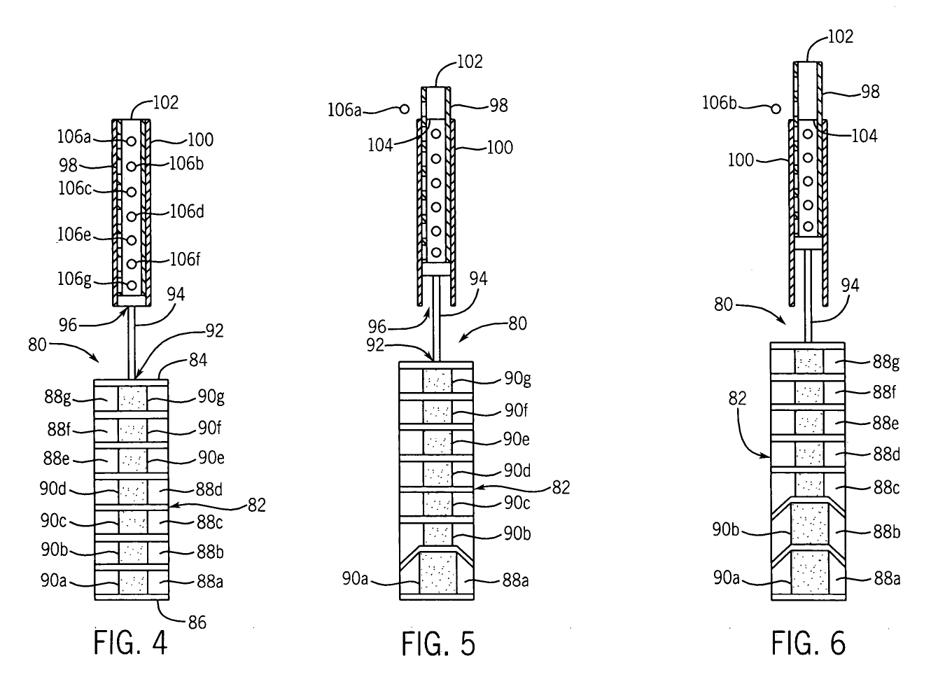

In operation, dispenser 80 is implanted within a human body at a user desired location that maximizes the beneficial effects of the dosages 106a-g of the microparticles. End 86 of actuator 82 is fixed with the body, as is outer sheath 100. In response to a predetermined stimuli exerted by a user, one of the hydrogel triggers 90a in section 88a of actuator 82 expands such that the overall length of actuator 82 increases. As the length of actuator 82 increases, plunger 94 moves axially into outer sheath 100 such that upper end 102 of inner sheath 98 separates from upper end 104 of outer sheath 100. An opening is provided in inner sheath 98 so as to allow dosage 106a to be released through the opening therein into the body, FIG. 5.

Referring to FIG. 6, in order to release dosage 106b , hydrogel trigger 90b in actuator section 88b is triggered by a user so as to expand and increase the overall length of actuator 82. Once again, as the length of actuator 82 increases, plunger 94 is urged further into outer sheath 100 such that upper end 102 of inner sheath 98 separates further from upper end 104 of outer sheath 100 thereby allowing second dosage 106b to be released into the human body through a corresponding opening in inner sheath 98. The process may be repeated such that the triggering of each remaining hydrogel trigger 90c-g results in a corresponding release of a dosage 106c-g, respectively, stored within inner and outer sheath 98 and 100, respectively.

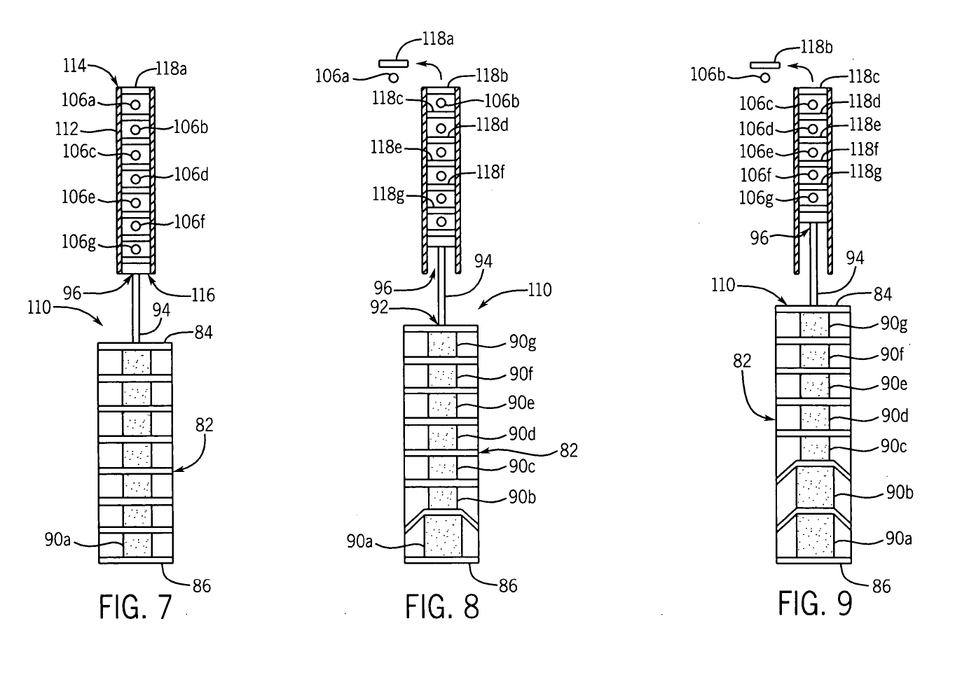

Referring to FIGS. 7-9, a third embodiment of a dispenser in accordance with the present invention is generally designated by the reference numeral 110. Dispenser 110 includes actuator 82 and plunger 94, as heretofore described. As such, the previous description of actuator 82 and plunger 94 with respect to dispenser 80 is understood to describe the actuator and the plunger of dispenser 110, as fully described herein.

Dispenser 110 further includes capillary 112 having a plurality of dosages 106a-g stored therein. Capillary 112 includes a first, open release end 114 for allowing dosages 106a-g to be dispensed into the human body therethrough, and a second open end 116 for receiving terminal end 96 of plunger 94. Each dosage 106a-g is associated with a corresponding impermeable segment 118a-g, respectively, disposed on the release end side of the dosage. Segments 118a-g prevent the premature dispensing of dosages 106a-g, respectively, as hereinafter described.

In operation, hydrogel trigger 90a expands in response to actuation by a user so as to increase the overall length of actuator 82. As trigger 90 expands, terminal end 96 of plunger 94 is urged into capillary 112 until such point as impermeable segment 118a and its corresponding dosage 106a are urged from release end 114 of capillary 112 into the human body, FIG. 8. With segment 118a and dosage 106a released from capillary 112, it can be appreciated that segment 118b prevents the premature dispensement of dosage 106b.

Referring to FIG. 9, in order to dispense dosage 106b, hydrogel trigger 90b is actuated by a user and expands, as heretofore described. As hydrogel trigger 90b expands, the length of actuator 82 increases and terminal end 96 of plunger 94 is urged further into the interior of capillary 112. As terminal end 96 of plunger 94 is urged further into the interior of capillary 112, segment 118b and corresponding dosage 106b are urged from release end 114 of capillary 112 into the human body, FIG. 9. It can be appreciated that by sequentially actuating hydrogel triggers 90c-g, dosages 106c-g will be sequentially released from the interior of capillary 112 through release end 114 thereof.

Once all of the dosages 106a-g are released from the interior of capillary 112, it is contemplated to reload capillary 112 with additional dosages of the microparticles. In addition, since expansion of the hydrogel triggers 90a-g is reversible, the hydrogel triggers 90a-g could be returned to their original size prior to the reloading capillary 112 with additional dosages of the microparticles.

Various modes of carrying out the invention are contemplated as being within the scope of the following claims particularly pointing out and distinctly claiming the subject matter, which is regarded as the invention.

1. A microparticle storage and delivery device for allowing an individual to selectively dispense a dosage of a microparticle, comprising:
a string loaded with a plurality of dosages of the microparticle;
a storage container for holding the string, the storage container having a first open end and a second opposite end;
a biasing structure for urging the string toward the open end; and
an actuator selectively engageable with the string to urge a dosage of the microparticle from the open end of the storage container.

2. The device of claim 1 wherein the string includes a hydrogel having the plurality of dosages contained therein.

3. The device of claim 1 further comprising a hollow actuator housing having an interior for slidably receiving the actuator therein and an open terminal end, the open end of the storage container communicating with the interior of the actuator housing.

4. The device of claim 3 wherein the interior of the actuator housing includes a staging section and a porous section, the open end of the storage container communicates with the staging section of the interior of the actuator housing.

5. The device of claim 4 further comprising a first valve for isolating the porous section from the staging section of the interior of the actuator housing.

6. The device of claim 5 further comprising a second valve for selectively closing the open end of the actuator housing.

7. The device of claim 1 further comprising a non-porous plug disposed in the storage container between the string and the biasing structure.

8. The device of claim 1 wherein second end of the storage container is porous.

9. The device of claim 1 wherein the biasing structure includes a hydrogel element, the hydrogel element expanding in response to a fluid passing through the second end of the storage container.

10. A dispenser implantable in the human body for allowing a user to selectively dispense a dosage of a microparticle, comprising:
a storage container for holding a plurality of dosages therein; and
an actuator operatively connected to the storage container, the actuator causing the release of a single dosage of the microparticle from the storage container in response to actuation by a user.

11. The dispenser of claim 10 wherein the actuator includes a series of hydrogel triggers, one of the hydrogel triggers expanding in response to actuation by the user.

12. The dispenser of claim 11 further comprising a plunger operatively engagable with the plurality of dosages, the plunger urging a single dosage from the storage container in response to expansion of the one of the hydrogel triggers.

13. The dispenser of claim 10 further comprising a string formed from a hydrogel, the hydrogel having the plurality of dosages of the microparticle contained therein.

14. The dispenser of claim 13 further comprising a biasing structure for urging the string from the storage container.

15. The dispenser of claim 14 further comprising a non-porous plug disposed in the storage container between the string and the biasing structure.

16. The device of claim 10 further comprising a hollow actuator housing having an interior for slidably receiving the actuator therein and an open terminal end and wherein the storage container has a first end communicating with the interior of the actuator housing and a second end.

17. The device of claim 16 wherein:
the interior of the actuator housing includes a staging section and a porous section; and
the storage container communicates with the staging section of the interior of the actuator housing.

18. The device of claim 17 further comprising:
a first valve for isolating the porous section from the staging section of the interior of the actuator housing; and
a second valve for selectively closing the open end of the actuator housing.

19. The device of claim 16 wherein second end of the storage container is porous.

20. A method of dispensing a dosage of a microparticle to an individual, comprising the steps of:
providing a dispenser;
loading the dispenser with a first plurality of dosages of the microparticle;
implanting the dispenser in a body; and
selectively releasing one of the first plurality of dosages.

22. The method of claim 21 comprising the additional step of encapsulating the first plurality of dosages of the microparticle in a hydrogel.

23. The method of claim 21 wherein the dispenser includes a storage container for receiving the first plurality of dosages therein.

24. The method of claim 23 wherein the step of selectively releasing one of the first plurality of dosages includes the additional step of urging a single dosage from the storage container.

25. The method of claim 21 comprising the additional step of isolating the first plurality of dosages of the microparticle in the dispenser from the interior of the body after the step of implanting the dispenser in a body.

26. The method of claim 21 comprising the additional step of reloading the dispenser with a second plurality of dosages of the microparticle after the first plurality of dosages is released into the body.
